(19) United States
(12) Patent Application Publication
(10) Pub. No.: US 2016/0184248 A1
(43) Pub. Date: Jun. 30, 2016

(54) COMPOSITIONS AND METHODS OF USE OF -HYDROXY-METHYLBUTYRATE (HMB) RESULTING IN AN ACUTE ENDOCRINE RESPONSE

(71) Applicant: University of Central Florida Research Foundation Inc., Orlando, FL (US)

(72) Inventors: Jay Hoffman, Oviedo, FL (US); John Rathmacher, Story City, IA (US); Jeffrey Stout, Orlando, FL (US); Jeremy Townsend, Orlando, FL (US); Shawn Baier, Ames, IA (US)

(21) Appl. No.: 14/969,662

(22) Filed: Dec. 15, 2015

Related U.S. Application Data
(60) Provisional application No. 62/092,009, filed on Dec. 15, 2014.

(51) Int. Cl. A61K 31/19

(52) U.S. Cl. CPC A61K 31/19

(57) ABSTRACT

The present invention provides a composition comprising HMB. Methods of administering HMB to an animal are also described. HMB is administered to induce an acute endocrine response. HMB is administered to increase circulating concentrations of growth hormone (GH) and insulin-like growth factor (IGF-1).

This application claims priority to U.S. Provisional Patent Application No. 62/092,009 filed Dec. 15, 2014 and herein corporates the provisional application by reference.

### BACKGROUND OF THE INVENTION

1. Field

The present invention relates to a composition comprising β-hydroxy-β-methylbutyrate (HMB) and methods of using HMB to result in an acute endocrine response. The acute endocrine response includes increasing circulating concentrations of growth hormone (GH) and insulin-like growth factor (IGF-1).

2. Background

HMB

In mammals and other higher order animals the first product of leucine metabolism is ketoisocaproate (KIC). A minor product of KIC metabolism is β-hydroxy-β-methylbutyrate (HMB). HMB has been found to be useful within the context of a variety of applications. Specifically, in U.S. Pat. No. 5,360,613 (Nissen), HMB is described as useful for reducing blood levels of total cholesterol and low-density lipoprotein cholesterol. In U.S. Pat. No. 5,348,979 (Nissen et al.), HMB is described as useful for promoting nitrogen retention in humans. U.S. Pat. No. 5,028,440 (Nissen) discusses the usefulness of HMB to increase lean tissue development in animals. Also, in U.S. Pat. No. 4,992,470 (Nissen), HMB is described as effective in enhancing the immune response of mammals. U.S. Pat. No. 6,031,000 (Nissen et al.) describes use of HMB and at least one amino acid to treat disease-associated wasting.

HMB is an active metabolite of the amino acid leucine. The use of HMB to suppress proteolysis originates from the observations that leucine has protein-sparing characteristics. The essential amino acid leucine can either be used for protein synthesis or transaminated to the α-ketoacid (α-ketoisocaproate, KIC). In one pathway, KIC can be oxidized to HMB. Approximately 5% of leucine oxidation proceeds via the second pathway. HMB is superior to leucine in enhancing muscle mass and strength. The optimal effects of HMB can be achieved at 3.0 grams per day, or 0.38 g/kg of body weight per day, while those of leucine require over 30.0 grams per day.

Once produced or ingested, HMB appears to have two fates. The first fate is simple excretion in urine. After HMB is fed, urine concentrations increase, resulting in an approximate 20-50% loss of HMB to urine. Another fate relates to the activation of HMB to HMB-CoA. Once converted to HMB-CoA, further metabolism may occur, either dehydration of HMB-CoA to MC-CoA, or a direct conversion of HMB-CoA to HMG-CoA, which provide substrates for intracellular cholesterol synthesis. Several studies have shown that HMB is incorporated into the cholesterol synthetic pathway and could be a source of cholesterol for new cell membranes that are used for the regeneration of damaged cell membranes. Human studies have shown that muscle damage following intense exercise, measured by elevated plasma CPK (creatine phosphokinase), is reduced with HMB supplementation within the first 48 hrs. post-exercise. The protective effect of HMB lasts up to three weeks with continued daily use. Numerous studies have shown an effective dose of HMB to be 3.0 grams per day as CaHMB (calcium HMB) (˜38 mg·kg body weight⁻¹·day⁻¹). This dosage increases muscle mass and strength gains associated with resistance training, while minimizing muscle damage associated with strenuous exercise (8; 16; 18; 20). HMB has been tested for safety, showing no side effects in healthy young or old adults. HMB in combination with L-arginine and L-glutamine has also been shown to be safe when supplemented to AIDS and cancer patients.

Recently, HMB free acid, a new delivery form of HMB, has been developed. This new delivery form has been shown to be absorbed quicker and have greater tissue clearance than CaHMB. The new delivery form is described in U.S. Patent Publication Serial No. 20120053240 which is herein incorporated by reference in its entirety.

A need exists for a composition and methods to induce an acute endocrine response, and to increase circulating concentrations of IGF-1 and/or GH. The present invention comprises a composition and methods of using a composition of HMB that results in such an increase in circulating concentrations of IGF-1 and/or GH and induces an acute endocrine response.

### SUMMARY OF THE INVENTION

One object of the present invention is to provide a composition that results in an acute endocrine response.

A further object of the present invention is to provide a composition for increasing circulating concentrations of growth hormone (GH).

Another object of the present invention is to provide a composition for increasing circulating concentrations of insulin-like growth factor (IGF-1).

An additional object of the present invention is to provide methods of administering a composition that result in an acute endocrine response and/or for increasing circulating concentrations of growth hormone (GH) and/or insulin-like growth factor (IGF-1).

A further object of the present invention is to provide methods of administering a composition for increasing calcium retention, improving bone density, improving bone strength, stimulating the immune system, improving cognitive function, improving insulin sensitivity and lowering blood glucose, reducing intrahepatic and intramyocellular lipids associated with insulin resistance, improving β-cell function of the pancreas, improving glucose-stimulated C-protein responses, attenuating type 2 diabetes, improving chronic liver disease, improving osteoporosis, having an antiaging effect on the heart and skeletal muscles, decreasing the incidence of heart failure, preserving satellite cells of the heart and skeletal muscle, preserving cardiac stem cells, decreasing the incidences of coronary heart disease, improving cardiovascular health, conserving protein during fasting, improving bone turnover, reducing fracture risk, slowing the progression of multiple sclerosis, improving the symptoms of Fibromyalgia Syndrome, improving the symptoms of Crohn's disease and ulcerative colitis, and improving cardiovascular disorders such as coronary disease, hypertension, heart failure and stroke.

The present invention intends to overcome the difficulties encountered heretofore. To that end, a composition comprising HMB is provided. The composition is administered to a subject in need thereof to result in an acute endocrine response, increase circulating concentrations of IGF-1 and/or GH, and improve the physiological, metabolic, endocrine and disease conditions described herein. All methods comprise administering to the animal HMB.

### BRIEF DESCRIPTION OF THE DRAWINGS

FIG. 1A is a graph showing plasma growth hormone concentrations for placebo (PL) and HMB free acid (HMB-acid or HMB-FA).

FIG. 1B is a graph showing area under the curve (AUC) for plasma growth hormone levels for PL and HMB-FA groups.

FIG. 2A is a graph showing plasma insulin-like growth factor (IGF-1) concentration values for placebo (PL) and HMB-FA groups.

FIG. 2B is a graph showing area under the curve (AUC) for plasma IGF-1 values for PL and HMB-FA groups.

### DETAILED DESCRIPTION OF THE INVENTION

The present invention comprises a composition of HMB and methods of use of HMB to result in an acute endocrine response, increased levels of IGF-1 and/or GH, and the associated results related to physiological, metabolic, endocrine and disease conditions. For example, administering HMB to a subject results in any of the following changes or improvements that are related to increases in IGF-1 and/or GH: for increasing calcium retention, improving bone density, improving bone strength, stimulating the immune system, improving cognitive function, improving insulin sensitivity and lowering blood glucose, reducing intrahepatic and intramyocellular lipids associated with insulin resistance, improving β-cell function of the pancreas, improving glucose-stimulated C-protein responses, attenuating type 2 diabetes, improving chronic liver disease, improving osteoporosis, having an antiaging effect on the heart and skeletal muscles, decreasing the incidence of heart failure, preserving satellite cells of the heart and skeletal muscle, preserving cardiac stem cells, decreasing the incidences of coronary heart disease, improving cardiovascular health, conserving protein during fasting, improving bone turnover, reducing fracture risk, slowing the progression of multiple sclerosis, improving the symptoms of Fibromyalgia Syndrome, improving the symptoms of Crohn's disease and ulcerative colitis, and improving cardiovascular disorders such as coronary disease, hypertension, heart failure and stroke.

The composition and methods of use of HMB described can be used on all age groups seeking the results identified herein.

The composition of HMB is administered to an animal in any suitable manner. Acceptable forms include, but are not limited to, solids, such as tablets or capsules, and liquids, such as enteral or intravenous solutions. Also, the composition can be administered utilizing any pharmaceutically acceptable carrier. Pharmaceutically acceptable carriers are well known in the art and examples of such carriers include various starches and saline solutions. In the preferred embodiment, the composition is administered in an edible form.

β-hydroxy-β-methylbutyric acid, or β-hydroxy-isovaleric acid, can be represented in its free acid form as (CH₃)₂(OH)CCH₂COOH. The term “HMB” refers to the compound having the foregoing chemical formula, in both its free acid and salt forms, and derivatives thereof. While any form of HMB can be used within the context of the present invention, preferably HMB is selected from the group comprising a free acid, a salt, an ester, and a lactone. HMB esters include methyl and ethyl esters. HMB lactones include isovalaryl lactone. HMB salts include sodium salt, potassium salt, chromium salt, calcium salt, magnesium salt, alkali metal salts, and earth metal salts.

Methods for producing HMB and its derivatives are well-known in the art. For example, HMB can be synthesized by oxidation of diacetone alcohol. One suitable procedure is described by Coffman et al., J. Am. Chem. Soc. 80: 2882-2887 (1958). As described therein, HMB is synthesized by an alkaline sodium hypochlorite oxidation of diacetone alcohol. The product is recovered in free acid form, which can be converted to a salt. For example, HMB can be prepared as its calcium salt by a procedure similar to that of Coffman et al. (1958) in which the free acid of HMB is neutralized with calcium hydroxide and recovered by crystallization from an aqueous ethanol solution. Currently, both the calcium salt of HMB and HMB as the free acid are commercially available from Metabolic Technologies, Ames, Iowa.

### Calcium β-hydroxy-β-methylbutyrate (HMB) Supplementation

More than 2 decades ago, the calcium salt of HMB was developed as a nutritional supplement for humans. Numerous studies have shown that CaHMB supplementation improves muscle mass and strength gains in conjunction with resistance-exercise training, and attenuates loss of muscle mass in conditions such as cancer and AIDS (1; 5; 16; 18; 20). Nissen and Sharp performed a meta-analysis of supplements used in conjunction with resistance training and found that HMB was one of only two supplements that had clinical studies showing significant increases in strength and lean mass with resistance training (18). Studies have shown that 38 mg of CaHMB per kg of body weight per day appears to be an efficacious dosage for an average person (8).

In addition to strength and muscle mass gains, CaHMB supplementation also decreases indicators of muscle damage and protein degradation. Human studies have shown that muscle damage following intense exercise, measured by elevated plasma CPK (creatine phosphokinase), is reduced with HMB supplementation. The protective effect of HMB has been shown to manifest itself for at least three weeks with continued daily use (8; 14; 16; 20) In vitro studies in isolated rat muscle show that HMB is a potent inhibitor of muscle proteolysis (19) especially during periods of stress. These findings have been confirmed in humans; for example, HMB inhibits muscle proteolysis in subjects engaging in resistance training (16).

The molecular mechanisms by which HMB decreases protein breakdown and increases protein synthesis have been reported (3; 22). Eley et al conducted in vitro studies which have shown that HMB stimulates protein synthesis through mTOR phosphorylation (2; 3). Other studies have shown HMB decreases proteolysis through attenuation of the induction of the ubiquitin-proteasome proteolytic pathway when muscle protein catabolism is stimulated by proteolysis inducing factor (PIF), lipopolysaccharide (LPS), and angiotensin II (3; 22-24). Still other studies have demonstrated that HMB also attenuates the activation of caspases-3 and -8 proteases (4). Taken together these studies indicate that HMB supplementation results in increased lean mass and the accompanying strength gains through a combination of decreased proteolysis and increased protein synthesis.

### HMB Free Acid Form

In most instances, the HMB utilized in clinical studies and marketed as an ergogenic aid has been in the calcium salt form (8; 16; 20). Recent advances have allowed the HMB to be manufactured in a free acid form for use as a nutritional supplement. Recently, a new free acid form of HMB was developed, which was shown to be more rapidly absorbed than CaHMB, resulting in quicker and higher peak serum HMB levels and improved serum clearance to the tissues (6; 7).

HMB free acid may therefore be a more efficacious method of administering HMB than the calcium salt form, particularly when administered directly preceding intense exercise. HMB free acid initiated 30 min prior to an acute bout of exercise was more efficacious in attenuating muscle damage and ameliorating inflammatory response than CaHMB. One of ordinary skill in the art, however, will recognize that this current invention encompasses HMB in any form.

HMB in any form may be incorporated into the delivery and/or administration form in a fashion so as to result in a typical dosage range of about 0.5 grams HMB to about 30 grams HMB.

Any suitable dose of HMB can be used within the context of the present invention. Methods of calculating proper doses are well known in the art. The dosage amount of HMB can be expressed in terms of corresponding mole amount of Ca-HMB. The dosage range within which HMB may be administered orally or intravenously is within the range from 0.01 to 0.5 grams HMB (Ca-HMB) per kilogram of body weight per 24 hours. For adults, assuming body weights of from about 100 to 200 lbs., the dosage amount orally or intravenously of HMB (Ca-HMB basis) can range from 0.5 to 30 grams per subject per 24 hours.

When the composition is administered orally in an edible form, the composition is preferably in the form of a dietary supplement, foodstuff or pharmaceutical medium, more preferably in the form of a dietary supplement or foodstuff. Any suitable dietary supplement or foodstuff comprising the composition can be utilized within the context of the present invention. One of ordinary skill in the art will understand that the composition, regardless of the form (such as a dietary supplement, foodstuff or a pharmaceutical medium), may include amino acids, proteins, peptides, carbohydrates, fats, sugars, minerals and/or trace elements.

In order to prepare the composition as a dietary supplement or foodstuff, the composition will normally be combined or mixed in such a way that the composition is substantially uniformly distributed in the dietary supplement or foodstuff. Alternatively, the composition can be dissolved in a liquid, such as water.

The composition of the dietary supplement may be a powder, a gel, a liquid or may be tabulated or encapsulated. Typically CaHMB is a powder than may be tabulated, encapsulate, or dissolved in liquid. HMB-acid is typically a liquid or gel that may be encapsulate, tabulated, or added to liquid.

Furthermore, the composition of the pharmaceutical medium can be intravenously administered in any suitable manner. For administration via intravenous infusion, the composition is preferably in a water-soluble non-toxic form. Intravenous administration is particularly suitable for hospitalized patients that are undergoing intravenous (IV) therapy. For example, the composition can be dissolved in an IV solution (e.g., a saline or glucose solution) being administered to the patient. Also, the composition can be added to nutritional IV solutions, which may include amino acids, peptides, proteins and/or lipids. The amounts of the composition to be administered intravenously can be similar to levels used in oral administration. Intravenous infusion may be more controlled and accurate than oral administration.

Methods of calculating the frequency by which the composition is administered are well-known in the art and any suitable frequency of administration can be used within the context of the present invention (e.g., one 3 g dose per day or two 1.5 g doses per day) and over any suitable time period (e.g., a single dose can be administered over a five minute time period or over a one hour time period, or, alternatively, multiple doses can be administered over an extended time period). HMB can be administered over an extended period of time, such as weeks, months or years.

Any suitable dose of HMB can be used within the context of the present invention. Methods of calculating proper doses are well known in the art. The dosage amount of HMB can be expressed in terms of corresponding mole amount of Ca-HMB. The dosage range within which HMB may be administered orally or intravenously is within the range from 0.01 to 0.2 grams HMB (Ca-HMB) per kilogram of body weight per 24 hours. For adults, assuming body weights of from about 100 to 200 lbs., the dosage amount orally or intravenously of HMB (Ca-HMB basis) can range from 0.5 to 30 grams per subject per 24 hours.

### Experimental Examples

The following examples further illustrate the invention but should not be construed as in any way limiting its scope. For example, the amount of HMB administered and the duration of the supplementation are not limited to what is described in the examples. The invention is also not limited to the particular form or type of HMB administered (CaHMB, HMB-acid, liquid, gel, powder, tablet, etc.).

### Methods

### Participants

Twenty resistance trained men (22.3±2.4 y, 1.8±0.1 m, 7.3±8.3 kg) volunteered to participate in this study. Participants were randomly separated into one of two groups: ingestion of HMB free-acid (HMB-FA; n=10) or ingestion of placebo (PL; n=10). Following an explanation of all procedures, risks, and benefits, each participant gave his informed consent prior to participation in this study. The Institutional Review Board for the protection of human subjects of the University of Central Florida approved the research protocol. For inclusion in this study, participants were required to have a minimum of one year of resistance training experience, particularly in the squat exercise. Participants were not permitted to use any additional nutritional supplements or medications while enrolled in this study. Screening for nutritional and hormonal supplements was accomplished via a health history questionnaire completed during participant recruitment.

### Study Protocol

The investigation utilized a placebo-controlled, double-blind, randomized design. Participants reported to the laboratory on two occasions. On the first visit (T1), participants were tested for their one-repetition maximum (1-RM) on the barbell back squat, dead lift, and barbell split squat exercises. Participants were instructed to refrain from any form of exercise for a minimum of 72 hours prior to the resistance training bout (T2). On T2, participants completed an intense lower-body resistance exercise session, which consisted of four sets of the barbell back squat, dead lift, and barbell split squat exercises. The barbell back squat exercise was performed with 80% of the participant's 1-RM and the dead lift and barbell split squat exercises were performed with 70% of the participant's 1-RM. Rest intervals were set at 90 s between each set and between exercises. Participants were encouraged to perform as many repetitions as possible up to 10 repetitions for each set. Total training volume, calculated as repetitions×load, was recorded for further analysis.

### HMB Free-Acid Supplementation

The HMB-FA supplement consisted of one gram of β-hydroxy-β-methylbutyrate in the free-acid form (Beta-TOR®, Metabolic Technologies Inc., Ames, Iowa), reverse osmosis water, de-bittering agent, flavor, stevia extract, and potassium carbonate. Each serving of placebo contained one gram of polydextrose and was identical to the HMB-FA supplement in appearance and taste. The HMB-FA and PL treatments were produced and supplied by Metabolic Technologies Inc. (Ames, Iowa). One serving of HMB-FA or PL was consumed 30 minutes prior to the exercise session. All HMB-FA and PL ingestion took place in the Human Performance Lab and was witnessed by one of the investigators.

### Anthropometric Measurements

Prior to maximal strength testing, anthropometric measurements, including height, body mass, and body fat percentage, were conducted. Body mass (±0.1 kg) and height (±0.1 cm) were measured using a Health-o-meter Professional (Patient Weighing Scale, Model 500 KL, Pelstar, Alsip, Ill., USA). All body composition measures were performed using standardized procedures previously described for collecting skinfold measurement from the triceps, suprailiac, abdomen, and thigh (11) and previously published formulas for calculating body fat percentage (13). All skinfold measurements were performed by the same researcher using the same skinfold caliper (Caliper-Skinfold-Baseline, Model #MDSP121110, Medline, Mundelein, Ill., USA).

### Blood Measurements

During the T2 experimental session, blood samples were obtained at pre-exercise (PRE), immediately post-exercise (IP), and 30 min post-exercise (30P). All blood samples were obtained using a 20-gauge Teflon cannula placed in a superficial forearm vein using a three-way stopcock with a male luer lock adapter. The cannula was maintained patent using an isotonic saline solution. PRE blood samples were drawn following a 15-min equilibration period prior to exercise. All IP blood samples were taken within one minute of exercise cessation. Following the resistance exercise protocol, subjects remained in the supine position for the full 30-min recovery phase prior to the 30P blood sample being drawn.

Blood samples were collected into two Vacutainer® tubes, one uncoated serum tube and one K₂EDTA plasma tube. A small aliquot of whole blood was removed from the K₂EDTA plasma tube and used for determination of hematocrit and hemoglobin. The blood in the serum tube was allowed to clot at room temperature for 30 minutes and subsequently centrifuged at 3,000×g for 15 minutes along with the remaining whole blood from the K₂EDTA plasma tube. The resulting plasma and serum was aliquoted into separate 1.8-mL microcentrifuge tubes and frozen at −80° C. for later analysis.

### Biochemical Analysis

Plasma HMB was analyzed by Metabolic Technologies Inc. by gas chromatography-mass spectrometry with previously outlined methods to confirm HMB appearance in the plasma (17). Myoglobin concentration was determined via a Myoglobin ELISA kit (Calbiotec, Cat no: MG017C, Spring Valley, Calif., USA) and prepared per manufacturer's instructions. Determination of serum immunoreactivity values was determined using a BioTek Eon spectrophotometer (BioTek, Winooski, Vt., USA). To eliminate inter-assay variance, all samples for a particular assay were thawed once and analyzed in the same assay run by a single technician. All samples were run in duplicate with a mean intra-assay variance of 5.73% for myoglobin.

Plasma testosterone (cat no: KGE010), Serum growth hormone (cat no: DGH00), and plasma IGF-1 (cat no: DG100) were assayed via commercial kits (R&D Systems Minneapolis, Minn., USA). Serum insulin was also assayed via a commercial kit (RayBiotech, Inc., Norcross, Ga., USA). Intra-assay variance for the hormones was 5.8%, 5.3%, 4.1%, 4.3% for testosterone, growth hormone, insulin, and IGF-1 respectively.

### Statistical Analysis

Prior to analysis, all data were assessed to ensure normal distribution, homogeneity of variance, and sphericity. A 2×3 analysis of variance (ANOVA) (group [PL, HMB-FA]×time [PRE, IP, 30P]) were used to analyze all biochemical data. When appropriate, follow-up analyses included one-way repeated measures ANOVAs and LSD post hoc comparisons. In the event PRE values were significantly different, and analysis of covariance (ANCOVA) to analyze the effects of the intervention. The area under the curve (AUC) for all hormone concentrations was calculated by using a standard trapezoidal technique and were analyzed using paired Student's t-tests. An alpha level of p<0.05 was used to determine statistical significance. All data are reported as mean±SD. Data were analyzed using SPSS v22 software (SPSS Inc., Chicago, Ill.).

### Results

The physical characteristics of the participants are presented in Table 1. No significant differences were noted in any in any of the anthropometric, strength and experience level characteristics between groups. Plasma HMB concentrations were significantly elevated at IP (p<0.01) and 30P (p<0.01) for HMB-FA only. In addition, the total training volume per exercise session was not statistically different between groups. The resistance exercise protocol resulted in significant elevations in myoglobin concentrations from PRE levels in both groups at IP (200.0%) and 30P (318.4%)”. Plasma HMB and myoglobin concentrations have been reported earlier (10).

TABLE 1
Characteristics of participants

| Characteristics | Placebo n = 10 | HMB-FA n = 10 |
|---|---|---|
| Age (years) | 23.8 ± 3.0 | 21.7 ± 2.0 |
| Height (m) | 1.78 ± 0.03 | 1.79 ± 0.08 |
| Weight (kg) | 85.7 ± 3.0 | 81.1 ± 12.7 |
| Body Composition (% fat) | 13.0 ± 3.0 | 13.1 ± 4.8 |
| Squat 1RM (kg) | 148.0 ± 3.0 | 135.9 ± 34.2 |
| Training experience (years) | 7.6 ± 3.0 | 5.95 ± 2.5 |

Values are means ± SD

### Hormone Responses

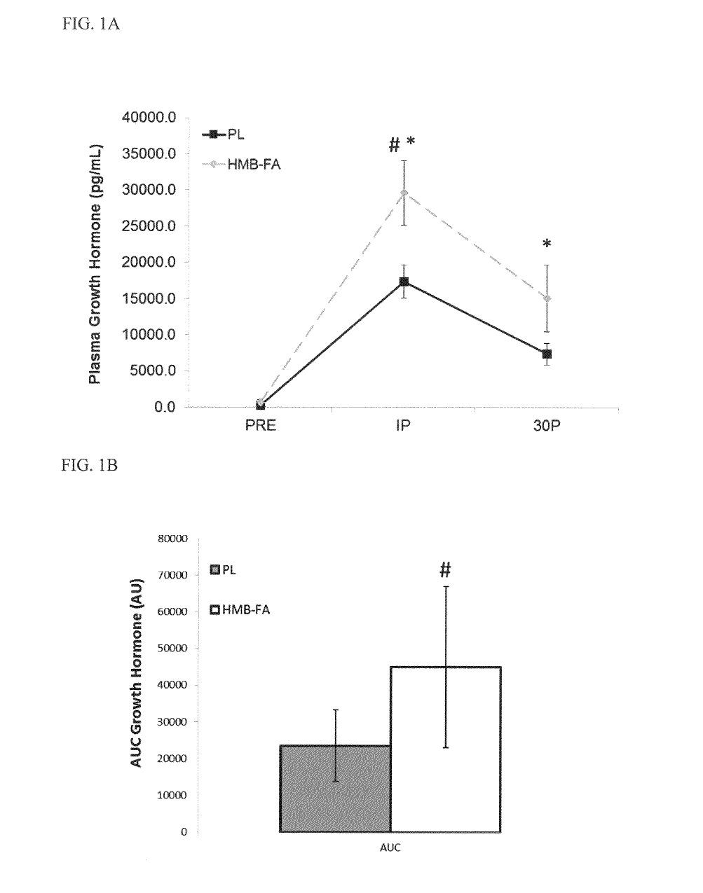

FIG. 1A shows plasma growth hormone concentration values for placebo (PL) and β-Hydroxy-β-methylbutyrate-Free Acid (HMB-FA) groups at Pre-exercise (PRE), Immediately post exercise (IP), 30-minutes post exercise (30P). Data are reported as mean±SD. * Indicates both groups were significantly elevated from PRE (p≦0.01). # Indicates the HMB-FA was significantly elevated from PL (p=0.05).

FIG. 1B shows area under the curve (AUC) analysis for plasma growth hormone levels for PL and HMB-FA groups. Data reported as mean±SD. # Indicates the HMB-FA group was significantly greater than PL (p=0.05).

Growth hormone concentrations were observed to increase from PRE at IP (p<0.001) and 30P (p<0.001) in both groups in response to the exercise protocol (FIG. 1A). However, the elevation in HMB-FA was significantly higher than PL at IP (p=0.021), but no difference between the groups were seen at 30P (p=0.100). AUC analysis revealed a significantly higher GH response (p=0.02) in the HMB-FA group compared to PL (see FIG. 1B)

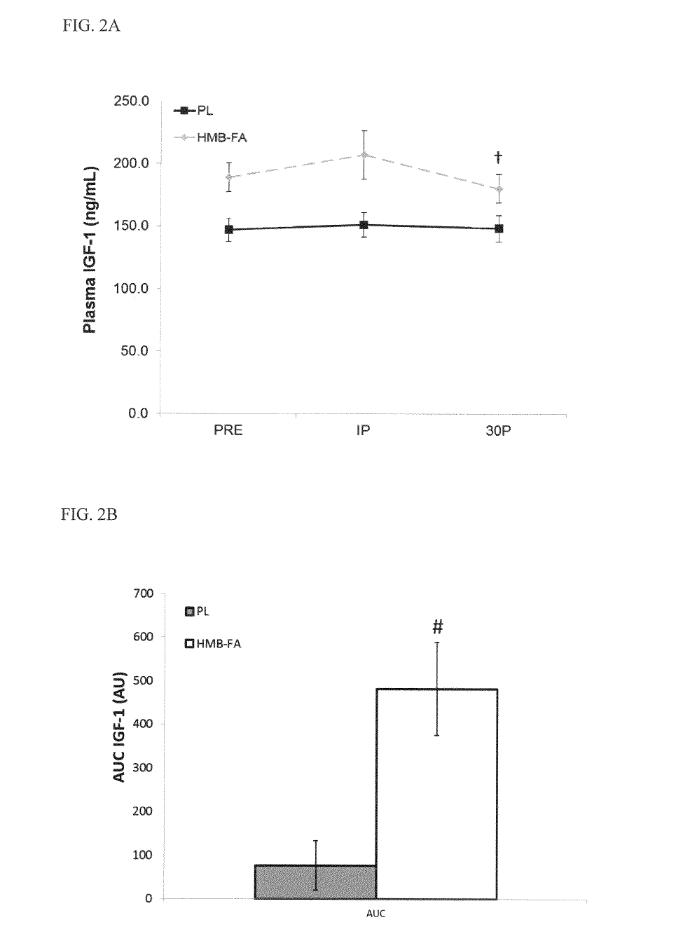

FIG. 2A shows plasma insulin-like growth factor (IGF-1) concentration values for placebo (PL) and β-Hydroxy-β-methylbutyrate-Free Acid (HMB-FA) groups at Pre-exercise (PRE), Immediately post exercise (IP), 30-minutes post exercise (30P). † Indicates both groups were significantly lower than PRE values.

FIG. 2B shows area under the curve (AUC) analysis for plasma IGF-1 levels for PL and HMB-FA groups. Data reported as mean±SD. # Indicates the HMB-FA group was significantly greater than the PL group (p=0.02).

Changes in IGF-1 concentrations from PRE to IP were not statistically different (p=0.69), but significantly declined from IP to 30P (p=0.015). IGF-1 values were significantly different (p=0.012) between the groups at PRE. ANCOVA results showed no differences in IGF-1 concentrations (p=0.31) in response to the workout. No differences were observed between the groups at IP or 30P (FIG. 2A). However, AUC analysis (see FIG. 2B) revealed a significant difference between HMB-FA and PL (p=0.02) with HMB-FA ingestion resulting in a greater IGF-1 response following the resistance training protocol compared to PL. There was a correlation trend between Plasma HMB AUC and IGF-1 AUC (r²=0.585; p=0.089).

### Discussion

This study demonstrates that HMB ingestion prior to resistance exercise can augment both the GH and IGF-1 response to a training session. Further, the study evidences a greater anabolic response associated with HMB supplementation.

There is a well-documented dose-response relationship between training volume and the concomitant elevation in growth hormone secretion (12; 15; 21; 25). The high volume and short rest period employed during this study elicited a significant elevation in the GH and IGF-1 response in both treatment groups. Despite the similar training volume between groups, HMB-FA ingestion immediately preceding the exercise protocol stimulated greater elevations in both GH and IGF-1 concentrations compared to PL.

The results of this study were unable to demonstrate any effect of HMB ingestion on the insulin response to the exercise protocol. This is in contrast with the study of Gerlinger-Romero and colleagues (9) who reported significant increases in resting insulin concentrations in HMB treated rats. These differences though are more likely a function of the prolonged supplementation protocol used by Gerlinger-Romero et al. (9), whereas we only investigated the hormonal responses from an acute ingestion and training session. While IGF-1 AUC values were significantly greater in the HMB-FA group in this study, the HMB-FA group was also elevated at baseline. Since the HMB-FA supplement was administered 15 min before the PRE blood draw, HMB-FA is promoting IGF-1 secretion independently of GH stimulation.

One gram of HMB-FA can promote a significantly greater post-exercise increase in GH and IGF-1 compared to PL. HMB modifies the GH/IGF-1 axis while promoting no differences in circulating testosterone levels.

Increases in GH and IGF-1 have significant implications for many physiological, metabolic, endocrine and disease conditions. For example, administering HMB to a subject results in any of the following changes or improvements that are related to increases in IGF-1 and/or GH: for increasing calcium retention, improving bone density, improving bone strength, stimulating the immune system, improving cognitive function, improving insulin sensitivity and lowering blood glucose, reducing intrahepatic and intramyocellular lipids associated with insulin resistance, improving β-cell function of the pancreas, improving glucose-stimulated C-protein responses, attenuating type 2 diabetes, improving chronic liver disease, improving osteoporosis, having an antiaging effect on the heart and skeletal muscles, decreasing the incidence of heart failure, preserving satellite cells of the heart and skeletal muscle, preserving cardiac stem cells, decreasing the incidences of coronary heart disease, improving cardiovascular health, conserving protein during fasting, improving bone turnover, reducing fracture risk, slowing the progression of multiple sclerosis, improving the symptoms of Fibromyalgia Syndrome, improving the symptoms of Crohn's disease and ulcerative colitis, and improving cardiovascular disorders such as coronary disease, hypertension, heart failure and stroke.

Thus, the findings that HMB supplementation increases IGF-1 and GH demonstrate that administration of a composition of HMB results in the improvements and changes listed above.

### REFERENCE LIST

1. Clark R H, Feleke G, Din M, Yasmin T, Singh G, Khan F and Rathmacher J A. Nutritional treatment for acquired immunodeficiency virus-associated wasting using β-hydroxy-β-methylbutyrate, glutamine and arginine: A randomized, double-blind, placebo-controlled study. JPEN J Parenter Enteral Nutr 24(3): 133-139, 2000.
2. Eley H L, Russell S T, Baxter J H, Mukerji P and Tisdale M J. Signaling pathways initiated by β-hydroxy-β-methylbutyrate to attenuate the depression of protein synthesis in skeletal muscle in response to cachectic stimuli. Am J Physiol Endocrinol Metab 293: E923-E931, 2007.
3. Eley H L, Russell S T and Tisdale M J. Attenuation of depression of muscle protein synthesis induced by lipopolysaccharide, tumor necrosis factor and angiotensin II by β-hydroxy-β-methylbutyrate. Am J Physiol Endocrinol Metab 295: E1409-E1416, 2008.
4. Eley H L, Russell S T and Tisdale M J. Mechanism of Attenuation of Muscle Protein Degradation Induced by Tumor Necrosis Factor Alpha and Angiotensin II by beta-Hydroxy-beta-methylbutyrate. Am J Physiol Endocrinol Metab 295: E1417-E1426, 2008.
5. Eubanks May P, Barber A, Hourihane A, D'Olimpio J T and Abumrad N N. Reversal of cancer-related wasting using oral supplementation with a combination of β-hydroxy-β-methylbutyrate, arginine, and glutamine. Am J Surg 183: 471-479, 2002.
6. Fuller J C, Jr., Sharp R L, Angus H F, Baier S M and Rathmacher J A. Free acid gel form of beta-hydroxy-beta-methylbutyrate (HMB) improves HMB clearance from plasma in human subjects compared with the calcium HMB salt. Br J Nutr 105: 367-372, 2011.
7. Fuller J C, Sharp R L, Angus H F, Khoo P Y and Rathmacher J A. Comparison of availability and plasma clearance rates of beta-hydroxy-beta-methylbutyrate delivery in the free acid and calcium salt forms. Br J Nutr 114: 1403-1409, 2015.
8. Gallagher P M, Carrithers J A, Godard M P, Schulze K E and Trappe S W. β-Hydroxy-β-methylbutyrate ingestion, Part I: Effects on strength and fat free mass. Med Sci Sports Exerc 32(12): 2109-2115, 2000.
9. Gerlinger-Romero F, Guimaraes-Ferreira L, Giannocco G and Nunes M T. Chronic supplementation of beta-hydroxy-beta methylbutyrate (HMB) increases the activity of the GH/IGF-I axis and induces hyperinsulinemia in rats. Growth Horm IGF Res 21: 57-62, 2011.
10. Gonzalez A M, Stout J R, Jajtner A R, Townsend J R, Wells A J, Beyer K S, Boone C H, Pruna G J, Mangine G T, Scanlon T M, Bohner J D, Oliveira L P, Fragala M S and Hoffman J R. Effects of beta-hydroxy-beta-methylbutyrate free acid and cold water immersion on post-exercise markers of muscle damage. Amino Acids 46: 1501-1511, 2014.
11. Hoffman J. Norms for Fitness, Performance, and Health. Champaign, Ill. USA: Human Kinetics, 2006.
12. Hoffman J R, Im J, Rundell K W, Kang J, Nioka S, Spiering B A, Kime R and Chance B. Effect of muscle oxygenation during resistance exercise on anabolic hormone response. Med Sci Sports Exerc 35: 1929-1934, 2003.
13. Jackson A S and Pollock M L. Practical assessment of body composition. Physician Sportsmed 13: 76-89, 1985.
14. Jówko E, Ostaszewski P, Jank M, Sacharuk J, Zieniewicz A, Wilczak J and Nissen S. Creatine and β-hydroxy-β-methylbutyrate (HMB) additively increases lean body mass and muscle strength during a weight training program. Nutr 17: 558-566, 2001.
15. Kraemer R R, Kilgore J L, Kraemer G R and Castracane V D. Growth hormone, IGF-I, and testosterone responses to resistive exercise. Med Sci Sports Exerc 24: 1346-1352, 1992.
16. Nissen S, Sharp R, Ray M, Rathmacher J A, Rice D, Fuller J C, Jr., Connelly A S and Abumrad N N. Effect of the leucine metabolite β-hydroxy β-methylbutyrate on muscle metabolism during resistance-exercise training. J Appl Physiol 81: 2095-2104, 1996.
17. Nissen S, Van Koevering M and Webb D. Analysis of β-hydroxy-β-methyl butyrate in plasma by gas chromatography and mass spectrometry. Anal Biochem 188: 17-19, 1990.
18. Nissen S L and Sharp R L. Effect of dietary supplements on lean mass and strength gains with resistance exercise: a meta-analysis. J Appl Physiol 94: 651-659, 2003.
19. Ostaszewski P, Kostiuk S, Balasinska B, Jank M, Papet I and Glomot F. The leucine metabolite 3-hydroxy-3-methylbutyrate (HMB) modifies protein turnover in muscles of the laboratory rats and domestic chicken in vitro. J Anim Physiol Anim Nutr (Swiss) 84: 1-8, 2000.
20. Panton L B, Rathmacher J A, Baier S and Nissen S. Nutritional supplementation of the leucine metabolite β-hydroxy β-methylbutyrate (HMB) during resistance training. Nutr 16: 734-739, 2000.
21. Pritzlaff C J, Wideman L, Weltman J Y, Abbott R D, Gutgesell M E, Hartman M L, Veldhuis J D and Weltman A. Impact of acute exercise intensity on pulsatile growth hormone release in men. J Appl Physiol (1985) 87: 498-504, 1999.
22. Russell S T and Tisdale M J. Mechanism of attenuation by beta-hydroxy-beta-methylbutyrate of muscle protein degradation induced by lipopolysaccharide. Mol Cell Biochem 330(1-2): 171-179, 2009.
23. Smith H J, Mukerji P and Tisdale M J. Attenuation of proteasome-induced proteolysis in skeletal muscle by β-hydroxy-β-methylbutyrate in cancer-induced muscle loss. Cancer Res 65: 277-283, 2005.
24. Smith H J, Wyke S M and Tisdale M J. Mechanism of the attenuation of proteolysis-inducing factor stimulated protein degradation in muscle by beta-hydroxy-beta-methylbutyrate. Cancer Res 64: 8731-8735, 2004.
25. Sutton J and Lazarus L. Growth hormone in exercise: comparison of physiological and pharmacological stimuli. J Appl Physiol 41: 523-527, 1976.

1. A composition comprising from about 0.5 g to about 30 g of β-hydroxy-β-methylbutyric acid (HMB), wherein administration of the composition to an animal in need thereof has the effect of increasing circulating concentrations of growth hormone (GH) and/or insulin-like growth factor (IGF-1).

2. The composition of claim 1, wherein said HMB is selected from the group consisting of its free acid form, its salt, its ester, and its lactone.

3. The composition of claim 2, wherein said salt is selected from the group consisting of a sodium salt, a potassium salt, a magnesium salt, a chromium salt and a calcium salt.

4. A method for increasing circulating concentrations of growth hormone (GH) and/or insulin-like growth factor (IGF-1) of an animal in need thereof comprising the steps of administering to said animal a composition of from about 0.5 g to about 30 g of β-hydroxy-β-methylbutyric acid (HMB), wherein upon said administration of said composition of HMB to the animal, GH and/or IGF-1 is increased.

5. The method of claim 4, wherein said HMB is selected from the group consisting of its free acid form, its salt, its ester, and its lactone.

6. The method of claim 5 wherein said salt is selected from the group consisting of a sodium salt, a potassium salt, a magnesium salt, a chromium salt and a calcium salt.

7. A method for inducing one or more of the physiological, metabolic, endocrine, or disease condition changes that result from increases in IGF-1 and/or GH in an animal in need thereof comprising the steps of administering to said animal a composition of from about 0.5 g to about 30 g of β-hydroxy-β-methylbutyric acid (HMB), wherein upon said administration of said composition of HMB to the animal, one or more of the physiological, endocrine, or disease condition changes are induced.

8. The method of claim 7, wherein the one or more changes induced are selected from the group consisting of increasing calcium retention, improving bone density, improving bone strength, improving osteoporosis, improving bone turnover and reducing fracture risk.

9. The method of claim 7, wherein the one or more changes induced are selected from the group consisting of stimulating the immune system and improving cognitive function.

10. The method of claim 7, wherein the one or more changes induced are selected from the group consisting of improving insulin sensitivity, lowering blood glucose, reducing intrahepatic and intramyocellular lipids associated with insulin resistance, improving β-cell function of the pancreas, improving glucose-stimulated C-protein responses, attenuating type 2 diabetes, and improving chronic liver disease.

11. The method of claim 7, wherein the one or more changes induced are selected from the group consisting of having an antiaging effect on the heart and skeletal muscles, decreasing the incidence of heart failure, preserving satellite cells of the heart and skeletal muscle, preserving cardiac stem cells, decreasing the incidences of coronary heart disease, improving cardiovascular health, conserving protein during fasting, and improving coronary disease, hypertension, heart failure and stroke.

12. The method of claim 7, wherein the one or more changes induced are selected from the group consisting of slowing the progression of multiple sclerosis, improving the symptoms of Fibromyalgia Syndrome, and improving the symptoms of Crohn's disease and ulcerative colitis.
